(19) United States
(12) Patent Application Publication
(10) Pub. No.: US 2018/0200433 A1
(43) Pub. Date: Jul. 19, 2018

(54) AUTOMATIC OPIOID ANTAGONIST INJECTION SYSTEM

(71) Applicant: Denis Baran Cirit, Laguna Niguel, CA (US)

(72) Inventor: Denis Baran Cirit, Laguna Niguel, CA (US)

(21) Appl. No.: 15/407,634

(22) Filed: Jan. 17, 2017

(51) Int. Cl. A61M 5/172; A61K 31/485; A61K 9/00

(52) U.S. Cl. CPC A61M 5/1723; A61K 31/485; A61K 9/0019; A61M 2205/3375; A61M 2205/3306; A61M 2230/42; A61M 2205/502; A61M 2205/52; A61M 2205/3592; A61M 2230/005; A61M 2230/04; A61M 2205/50

(57) ABSTRACT

A system for automatically injecting naloxone into a patient, upon detection of physiological parameters indicative of an opioid overdose.

### FIELD OF THE INVENTIONS

The inventions described below relate to the field of treatment for opioid overdose.

### BACKGROUND OF THE INVENTIONS

According to the Center for Disease Control, opioid overdose killed 28,647 people in the United States in 2014 and 33,091 in 2015. This is several times the number of deaths in 2000. Every day in America, over 1,000 people are treated in emergency departments for misusing prescription opioids. Other countries, such as Canada, are experiencing similar death rates, and increases in death rates, due to opioid overdose. Worldwide, 69,000 people die from opioid overdose each year. Opioids include natural and semisynthetic opioids such as morphine, heroin, tramadol, oxycodone, hydrocodone and synthetic opioids such as methadone and fentanyl. An overdose of opioids results in reduced breathing rates (respiratory depression) and greatly reduced heart rate (bradycardia), which can both be lethal.

Naloxone (NARCAN®), an opioid antagonist, is an antidote to opioid overdose, and will completely reverse the effects of an opioid overdose immediately, and save the life of an overdose victim. Naloxone is effective when delivered by intravenous injection, intramuscular injection (into a muscle), subcutaneous injection (under the skin), and intranasal spray. Naloxone has no detrimental or beneficial effect on people who have not taken opioids. For this reason, it is the preferred antidote drug for treating opioid overdose. Other opioid antagonists include nalmefene (SELINCRO®, approved in the United States as an antidote), nalorphine (which is disfavored due to its side effects), and naltrexone.

The World Health Organization recommends that naloxone be made available to people likely to witness an opioid overdose, but not all overdoses are witnessed. Naloxone is available subject to restrictions which vary from state to state, and country to country. Efforts to make naloxone accessible to overdose victims are dependent on witnesses to diagnose an overdose and inject naloxone. An unwitnessed overdose is likely to go untreated, and the unwitnessed overdose victim is likely to die. Nonetheless, those likely to overdose on opioids presumably know that they are addicted and subject to overdose, and could take precautions to avoid the dangers of overdose, if such precautions were available. Also, in hospital settings, quicker diagnosis and treatment of opioid overdose would be beneficial.

### SUMMARY

The systems and methods described below provide for automatic administration of naloxone to an opioid overdose victim. The system comprises an injector for injecting an opioid antagonist, a sensor for sensing a physiological parameter such as breathing rate or heart rate, which may be indicative of opioid overdose, and a controller. The controller is programmed and operable to interpret the sensor signals corresponding to the physiological parameter and, upon determining that the physiological parameter is in a range indicative of an opioid overdose, operate the injector to inject a therapeutically effective dose of naloxone subcutaneously into the patient. The system may be configured in a single device, such as a wristband or ankle bracelet, which may be applied to the skin of an opioid user, overlying a portion of the body from which the user's breathing rate or heart rate or other physiological parameter can be sensed, and into which the naloxone can be injected, or the system may be configured as a system with separate sensor, injector, and controller components, with the sensor located at an optimal sensing location and the injector located at an optimal injection site. Other opioid antagonists may be used in place of naloxone.

### BRIEF DESCRIPTION OF THE DRAWINGS

FIG. 1 is a schematic of the system for automatically injecting naloxone into a patient in response to a physiological parameter indicative of an opioid overdose.

FIG. 2 illustrates a convenient embodiment of the system of FIG. 1.

FIGS. 3 and 4 illustrate use of the device of FIG. 2.

### DETAILED DESCRIPTION OF THE INVENTIONS

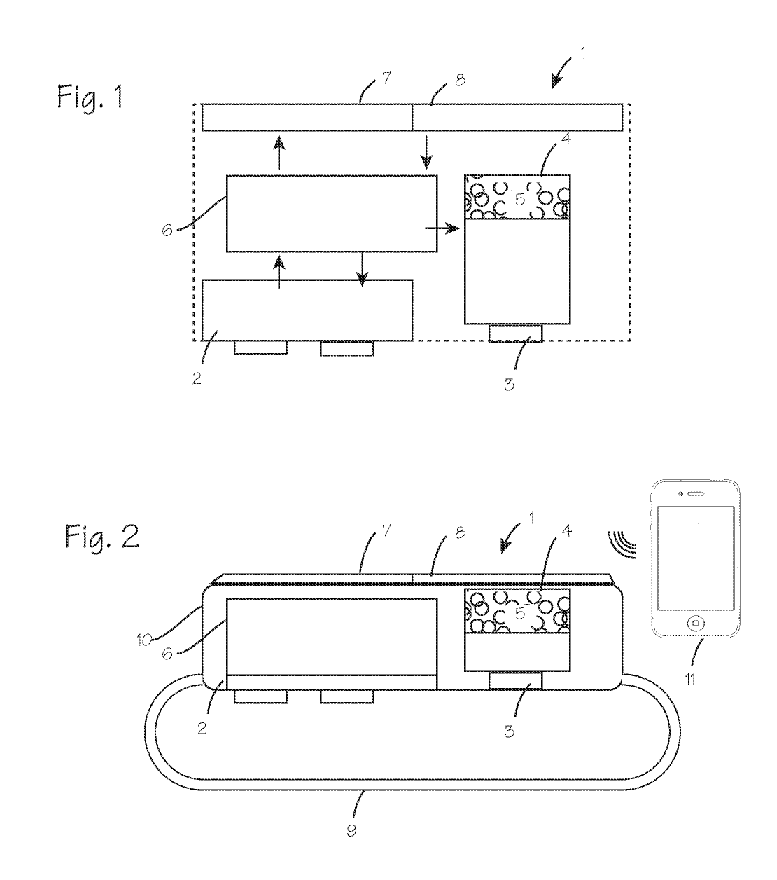

FIG. 1 is a schematic of the device for automatically injecting naloxone into a patient in response to detection of a physiological parameter indicative of an opioid overdose. The system 1 includes a sensor assembly 2 operable to sense a physiological parameter such as the breathing rate or heart rate of a patient, an injector assembly 3 in fluid communication with a reservoir 4 filled, or to be filled, with an opioid antagonist 5, and a control system 6 which is operable and programmed to (1) receive signals from the sensor, determine from the signals the breathing rate or heart rate or other physiological parameter of the patient, and, if the determined breathing rate or heart rate or other physiological parameter falls within a range indicative of an opioid overdose (2) operate the injector to inject the opioid antagonist into the patient. The system may also include a user output assembly 7, which may be any form of display, and user input assembly 8.

FIG. 2 illustrates a convenient embodiment of the device of FIG. 1. In FIG. 2, the system 1 is configured as a wrist-band, with sensor 2, the reservoir 4, the injector 3, and the control system 6 mounted on the band 9. The various components may be conveniently disposed in a housing 10, or may be placed in different locations on or off the band. For example, the sensor, or sensing components of the sensor, may be disposed on the strap, displaced from the housing, such that the housing may be held on one side of the distal forearm above the wrist (the dorsal side, like a watch) while the sensing components of the sensor are held on the ventral side, over the pulse point of the lower forearm just above the wrist. The device may include the display, operable by the control system, to provide a user interface for controlling the device, and presenting information to a user or third party. Control system components necessary for calculating the detected breathing rate or heart rate, controlling the display, controlling the device, etc. may be housed in an external device 11 (a smartphone, for example), rather than the housing, in which case the system may include a transceiver coupled to the sensor and injector for passing control and operation information back and forth between the system and the external device, and the display of the external device may be used in place of the user output 7.

For detecting breathing rate, the sensor may comprise a respiratory sensor (contact, non-contact, or acoustic) operable to detect the respiratory rate of the opioid user. A photoplethysmographic sensor, and ECG sensor, accelerometers, an end-tidal CO₂ detector, a noninvasive acoustic respiration rate detector, or remote means of sensing respiration may be used. In the case of the contact or non-contact respiration sensor, a respiratory rate below twelve breaths per minute may be used as the indication of an opioid overdose.

For detecting heart rate, the pulse sensor comprises a photoplethysmographic sensor, and ECG sensor, or other sensor operable to detect the heart rate. Current photoplethysmographic pulse sensors can detect a patient's heart rate from most any location on the surface of the body. Commercially available photoplethysmographic pulse sensors are mounted on wrist bands, chest straps, and fingertip clips. Wrist-mounted photoplethysmographic pulse sensors are operable to sense the pulse from the dorsal side of the lower forearm just above the wrist or the ventral side of the lower forearm just above the wrist, and disposed on straps to hold the sensors to the forearm. These may be used in the system described in relation to FIGS. 1 and 2. The sensor and mounting structure may be configured to be held in contact with a pulse point on the body, for more certain pulse detection.

The system may use various other sensors to detect physiological parameters as a basis for automatically injecting naloxone into a patient in response to an opioid overdose, and the system may be generalized to operate with physiological parameters other that respiration rate or heart rate, using other sensors. For example, a pulse oximeter (photoplethysmographic sensor) operable to detect blood oxygen levels (SpO₂ or PaO₂ or other measure) may be used to determine that the patient is hypoxic, or a blood CO₂ (PaCO₂) sensor operable to detect CO₂ levels in the opioid user may be used. In the case of the pulse oximeter, a blood oxygen level well below the normal range of 95-100% (SpO₂) or 80 mmHg (PaO₂), may be used as an indication of hypoxemia that is indicative of an opioid overdose. For example, a blood oxygen level (SpO₂) of below 90% or 80% may be used as the predetermined range for this physiological parameter that is indicative of an opioid overdose. In the case of CO₂ detector, a carbon dioxide level, such as PaCO₂, well above the normal range of 35-45 mmHg (4.7-6.0 kPa), may be used as the predetermined range for this physiological parameter, as an indication of hypercapnia that is indicative of an opioid overdose. The system may employ only one sensor and determine that the patient is experiencing an opioid overdose based on the value of the corresponding physiological parameter, or the system may use any combination of sensors and the values of the corresponding physiological parameters to make the determination.

The injector and reservoir may be any form of injector, and the reservoir may have a capacity sufficient to hold a therapeutically effective dose of the chosen antagonist. The injector may be operated with a spring mechanism acting on a piston in the reservoir, or operated with a pump in fluid communication between the reservoir and the injection needle, or any other mechanism for injecting the antagonist. The injector includes a needle of sufficient length and gauge to quickly inject the antagonist into the patient to achieve intravenous injection, intramuscular injection or subcutaneous injection. Subcutaneous injection at the lower forearm, for example, will be effective. Multiple injectors and/or reservoirs may be installed in the system, and the reservoir may be large enough to hold several doses of an antagonist.

Naloxone (NARCAN®) is the opioid antagonist currently preferred for use with the system, as it can completely reverse the effects of an opioid overdose immediately and has no side effects of significant concern. Naloxone is effective when delivered by intravenous injection, intramuscular injection (into a muscle), subcutaneous injection (under the skin), and intranasal spray. Nalmefene, nalorphine, and naltrexone may be used, despite minor side effects they may have. Any other opioid antagonists may be used, even if they have significant side effects. The therapeutically effective dose of any of these antagonists is small, and can be accommodated in a reservoir which fits in a small housing comparable to a wristwatch. A therapeutically effective dose of naloxone is 0.4 mg to 2 mg (initial dose) with repeated doses as necessary at two or three minute intervals. A therapeutically effective dose of Nalmefene is (1.5 mg), or 0.25 mcg/kg intravenously once as an initial dose, followed by 0.25 mcg/kg incremental doses at 2 to 5 minute intervals until the desired degree of opioid reversal is obtained. A therapeutically effective dose of nalorphine is 5-10 mg repeated every 10-15 minutes as required until respiration is restored, to a maximum dose of 40 mg. Larger doses will also be therapeutic doses, because the side effects of the drugs are inconsequential compared to the consequence of the overdose, and precise dosing is unnecessary.

The control system is preferably a computerized control system which comprises at least one processor and at least one memory including program code configured with the processor to cause the system to perform the functions described throughout this specification. The control system is operably connected to the sensor and the injector, and is operable to receive the signals from the sensor corresponding to the physiological parameter (respiration rate or heart rate of the patient or other physiological parameter) and operable to control the injector to inject opioid antagonist into the patient. The control system is programmed to determine if the signals corresponding to the physiological parameter are indicative of an opioid overdose, and further programmed such that, upon determining that the signals corresponding to the heart rate or other physiological parameter are indicative of an opioid overdose, generate control signals and transmit those signals to the injector to cause the injector to inject a therapeutically effective dose of the opioid antagonist into the patient. The control system will be programmed to cause injection of antagonist when the physiological parameter falls within the predetermined range, as determined by analysis of the signals from the sensor. The predetermined range may be a respiration rate below 12 breaths per minute, or a heart rate below 40 beats per minute. Instantaneous rates may be used, several repeated measurements over a short period may be used, or a sustained period of reduced rates (for example, at least thirty seconds), may be used to confirm that the physiological parameter is within the predetermined range. The algorithm for deciding that an overdose has occurred, and the predetermined ranges of the measured physiological parameter, may be modified as clinical experience dictates. The control system may rely on a single physiological parameter, or a combination of physiological parameters, in determining that predetermined conditions for injection into the patient have been met.

The control system may be further programmed for additional functionality. For example, the control system may be programmed to continue monitoring the patient's respiration rate or heart rate, immediately after causing a first injection, and, upon detecting a continued low respiration rate or heart rate, cause the injector to inject an additional dose of antagonist, or upon detecting a higher respiration rate or heart rate (indicative of recovery), securing the injector. The control system may also operate the user output to present indicators regarding the system status. For example, if the user output is a display screen, the control system can operate the display to indicate that an opioid overdose was detected, that an antagonist dose was injected, and to present the current respiration rate or heart rate or current status of the physiological parameter used to detect an overdose. The control system may also be programmed to operate the display to indicate that an opioid overdose was detected, operate the display to prompt a user to provide input to direct the system to inject the opioid antagonist, and receive input from a user input, and inject the opioid antagonist in response to a manually applied input from a user or attendant.

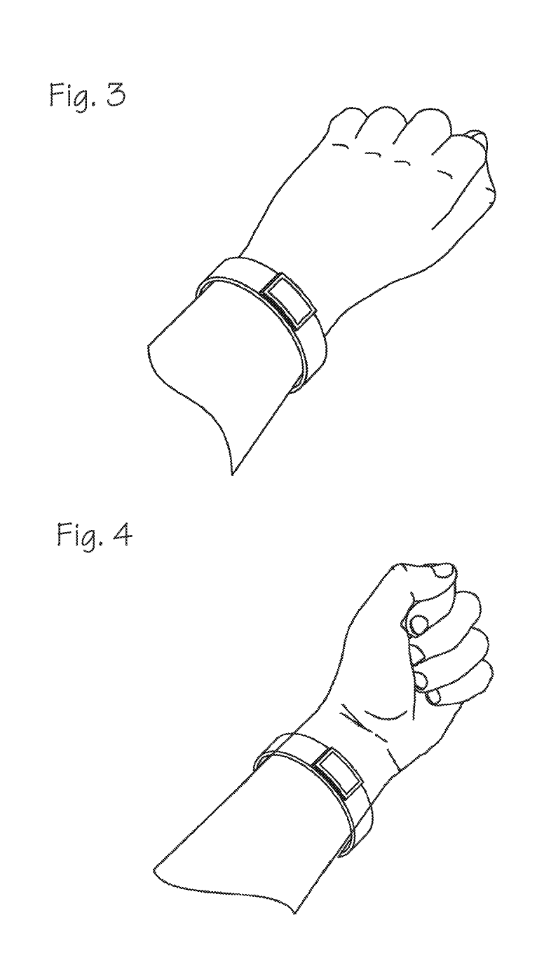

In use, the device may be provided by prescription, or over-the-counter, to opioid users. The device may be used in clinical, hospital, or surgical settings, as a preventive measure for patients taking opioids to treat pain from surgery or illness. The device may also be used as a preventive measure for opioid addicts. A user or attendant will apply the device to the body of the user, turn the device on, and continue with appropriate or inappropriate use of the opioid. This will cause the system to monitor the patient's physiological parameter continuously, and, upon detecting a physiological parameter in a range indicative of an overdose (according to its algorithm), operate the injector to inject the antagonist. The user may apply the device to the lower forearm just above the wrist, for example, as shown in FIGS. 3 and 4, applying the sensors and injector to either the dorsal side of the distal forearm (FIG. 3) or the ventral side of the distal forearm (FIG. 4), just above the wrist.

The mounting structures may be configured as watchbands or watches, bracelets, ankle bracelets, armbands, chest straps, headbands, necklaces or the like, or adhesive bandages. Mounting structures may also include such things as eyeglass frames to hold the sensor over the temporal artery on the side of the forehead, tightly fitting shirts to hold the sensor over the xiphoid process, and finger-tip clips, or a glove or strap to hold the sensor over the pad of the thumb.

The system may generally be described as comprising (1) a sensor operable to detect, in the patient, a physiological parameter affected by an opioid overdose and generate signals corresponding to the physiological parameter; (2) a reservoir of an opioid antagonist, and an injector operable to inject opioid antagonist into the patient; and (3) a control system, operably connected to the sensor and the injector. The control system is programmed to be operable to receive the signals corresponding to the physiological parameter and operable to control the injector to inject opioid antagonist into the patient and to determine if the signals corresponding to the physiological parameter are indicative of an opioid overdose. The control system is further programmed such that, upon determining that the signals corresponding to the physiological parameter are indicative of an opioid overdose, the control system will cause the injector to inject a therapeutically effective dose of the opioid antagonist into the patient.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

I claim:

1. A system for injecting an opioid antagonist into a patient in response to a physiological parameter indicative of an opioid overdose, said system comprising:
a sensor operable to detect, in the patient, a physiological parameter affected by an opioid overdose and generate signals corresponding to said physiological parameter;
a reservoir of an opioid antagonist, and an injector operable to inject opioid antagonist into the patient;
a control system, operably connected to the sensor and the injector, said control system operable to receive the signals corresponding to the physiological parameter and operable to control the injector to inject opioid antagonist into the patient, said control system programmed to determine if the signals corresponding to the physiological parameter are indicative of an opioid overdose, said control system further programmed such that, upon determining that the signals corresponding to the physiological parameter are indicative of an opioid overdose, the control system causes the injector to inject the opioid antagonist into the patient.

2. The system of claim 1, wherein the sensor is a respiration rate sensor, operable to detect the patient's respiration rate, and the control system is programmed to determine that a respiration rate below twelve breaths per minute as indicative of an opioid overdose.

3. The system of claim 1, wherein the sensor is a photoplethysmographic sensor operable to detect the patient's respiration rate.

4. The system of claim 1, wherein the sensor is ECG sensor operable to detect the patient's respiration rate.

5. The system of claim 1, wherein the sensor is a respiration rate sensor, operable to detect the patient's respiration rate, and the control system is programmed to determine that a respiration rate below twelve breaths per minute, over a period of at least thirty seconds, as indicative of an opioid overdose.

6. The system of claim 1, wherein the sensor is an acoustic sensor.

7. The system of claim 1, wherein the sensor is a non-contact sensor.

8. The system of claim 1, wherein the sensor is secured to a mounting means configured to hold the sensor proximate to the body of the patient.

9. The system of claim 1, wherein the sensor is secured to a band configured to hold the sensor in contact with a pulse point on the body of the patient.

10. The system of claim 1, wherein the sensor, injector, and control system are disposed within a single housing, and said housing is secured to a mounting structure configured to hold the housing proximate a pulse point on the patient's body.

11. The system of claim 1, wherein the sensor and injector are disposed in a single housing, and the controller is disposed in a separate device external to the housing.

12. The system of claim 1, wherein the sensor and injector are disposed in separate housings, and the sensor is secured to a first mounting structure configured to hold the sensor proximate a sensing point on the body of the patient, the injector is secured to a second mounting structure configured to hold the injector proximate an injection site on the body of the patient.

13. The system of claim 1, wherein the sensor is a pulse sensor, operable to detect the patient's heart rate, and the control system is programmed to determine that a heart rate below 40 beats per minute as indicative of an opioid overdose.

14. The system of claim 1, wherein the sensor is a photoplethysmographic sensor, operable to detect the patient's heart rate.

15. The system of claim 1, wherein the sensor is an ECG sensor, operable to detect the patient's heart rate.

16. The system of claim 1, wherein the sensor is a pulse sensor, operable to detect the patient's heart rate, and the control system is programmed to determine that a heart rate below 40 beats per minute, over a period of at least thirty seconds, as indicative of an opioid overdose.

17. The system of claim 13, wherein the sensor is secured to a band configured to hold the sensor in contact with a pulse point on the body of the patient.

18. The system of claim 13, wherein the sensor, injector, and control system are disposed within a single housing, and said housing secured to a mounting structure configured to hold the housing proximate a pulse point on the patient's body.

19. A method of treating opioid overdose in a patient, said method comprising:
disposing a sensor operable to detect a physiological parameter of the patient affected by an opioid overdose, proximate the body of the patient;
disposing an injector proximate an injection site on the body of the patient, said injector operable to inject an opioid antagonist;
operating a controller to obtain signals corresponding to physiological parameter of the patient, and operating the controller to inject opioid antagonist into the patient when the signals corresponding to physiological parameter of the patient correspond to a physiological parameter indicative of an opioid overdose.

20. The method of claim 19, wherein:
the sensor is a pulse sensor.

21. The method of claim 19, wherein:
the sensor is a respiration rate sensor.

22. The method of claim 19, wherein:
the sensor is a photoplethysmographic sensor.
